(19) United States
(12) Patent Application Publication
(10) Pub. No.: US 2016/0228271 A1
(43) Pub. Date: Aug. 11, 2016

(54) Implantable Medical Devices

(71) Applicant: RAINBOW MEDICAL ENGINEERING LIMITED, Royston (GB)

(72) Inventors: Alan Lynford Profit, Essex (GB); Luigi Gerard Martini, London (GB)

(21) Appl. No.: 15/023,019

(22) PCT Filed: Sep. 19, 2014
(86) PCT No.: PCT/GB2014/052854

§ 371 Date: Mar. 18, 2016

(30) Foreign Application Priority Data
Sep. 20, 2013 (GB) 1316738.2

(51) Int. Cl. A61F 2/91; B26D 7/08; B29C 71/02; B29C 47/00; A61L 31/04; A61L 31/14

(52) U.S. Cl. CPC A61F 2/91; A61L 31/041; A61L 31/14; B29C 71/02; B29C 47/0066; B26D 7/086; A61F 2240/001; A61F 2210/0014; A61F 2210/0071; A61F 2250/0067; B29C 2071/022

(57) ABSTRACT

There is described a method of manufacturing a polymeric implantable medical device wherein the medical device comprises a tube with an opening said method comprising the simultaneous cutting and annealing of the opening; and polymeric implantable medical devices.

### FIELD OF THE INVENTION

The present invention relates to a novel method of manufacturing implantable medical devices

More particularly, the present invention relates to a novel method of manufacturing implantable medical devices, such as endoprostheses, and to implantable medical devices prepared by said method.

### BACKGROUND OF THE INVENTION

There are a number of medical conditions that may require a medical device to be implanted into a patient's body. One example of such a condition is stenosis, narrowing or constriction of the diameter of a bodily passage or orifice, e.g. atherosclerotic stenosis in blood vessels. In the treatment of stenosis a medical device, usually artificial implantable medical device, such as, an endoprosthesis, or stent, is placed inside the body of a patient.

However, one problem encountered with the use of artificial implantable medical device is that a thin biofilm can be formed. In such percutaneous interventional procedures the body surface of the patient must be penetrated and unwanted bacteria, etc. may enter the body via the penetrating region, giving rise to a risk of infection.

Artificial implantable medical devices, such as, an endoprosthesis, or stent, will usually comprise a thin tube, usually extruded from medical grade materials, and provided with at least one opening or “eye hole”. The existing manufacturing process for endoprostheses is crude, resulting in surface imperfections in the endoprostheses being common, particularly adjacent to the “eye hole” of the endoprostheses increasing the exposure to infecting bacteria.

The conventional process for manufacturing endoprostheses comprises punching or rupturing a hole into an endoprosthesis tube. However, this method results in a rough surface around the opening of the hole. Furthermore, it has been found that the rough surface enables infectious microorganisms, such as, bacteria and/or yeasts, to attach to the roughened surface of the endoprosthesis which results in the formation of a biofilm of bacteria and/or yeast, which can introduce infections to the patient and/or make the treatment of an infection considerably more difficult.

US Patent application No. 2003/158540 describes a method for reducing the incidence of urinary tract infection in a patient having an indwelling catheter by using a weak acidic solution (acetic acid) to treat the catheter.

Martini L. G., et al “A Cut Above in Urinary Catheter Fabrication” European Medical Device Technology, available at http://www.emdt.co.uk/article/cut-above-urinary-catheter-fabrication describes the ultrasonic aperture cutting of drainage holes in urinary catheters which provide reduced encrustation when exposed to artificial urine.

European Patent No. EP 1920724 describes the use of a circular RF electrode for in situ cutting a side opening in a stent-graft material.

Therefore, there is a need for an improved method of manufacturing medical devices that require cutting, which overcomes or mitigates the disadvantages of conventional prior art methods.

### SUMMARY OF THE INVENTION

It has been surprisingly found that the topology of the endoprosthesis surface can have an impact on the ability of bacteria to form biofilms, which can seriously complicate the care of patients. The roughness or rugosity of the endoprosthesis substrate is known to play a significant role in the bacterial/yeast attachment process, particularly when the surface irregularities are comparable to the size of the bacteria/yeast and can provide shelter from unfavourable environmental factors.

We have now found a novel method of manufacturing an implantable medical device, e.g. an implantable medical device which comprises a polymeric material, such as an endoprosthesis, etc. which provides an implantable medical devices with smoother finish of lower rugosity, particularly adjacent the opening or “eye hole”.

Thus, according to a first aspect of the invention there is provided a method of manufacturing a polymeric implantable medical device wherein the medical device comprises a tube with an opening said method comprising the simultaneous cutting and annealing of the opening, e.g. in the tube.

Although a variety of cutting methods may suitably be employed for the simultaneous cutting and annealing of the opening of the polymeric implantable medical device, a preferred process comprises ultrasonically cutting an opening in the polymeric implantable medical device.

The process according to this aspect of the invention will generally comprise the use of an ultrasonic drill or scalpel, which may be known as an ultrasound horn or sonotrode. Ultrasonic drills, scalpels, horns and sonotrodes are commercially available and generally comprise a boring or cutting device that uses vibrations in order to drill through materials. The shape of the ultrasound horn or sonotrode may vary, depending upon the nature of the device being manufactured. For example, the ultrasound horn or sonotrode may be shaped in such a way that it may cut the device whilst simultaneously removing any debris from the cutting process. Furthermore, the shape of ultrasound horn or sonotrode may be such that the maximum energy is channelled to the tip of the horn or sonotrode.

The specific characteristics of the drill make it ideal or practical for certain situations. For example, the ultrasonic drill may be driven by a piezoelectric actuator that creates the vibrations at an extremely high frequency. The ultrasonic drill will generally have low axial load and ensures that no damage is done to the polymeric implantable medical device and a smooth, low rugosity opening is achieved. An ultrasound scalpel comprises an ultrasound generator, ultrasound transducer, transducer tip, and a cavity on the transducer tip using a liquid spray shaped to form a cutting surface. The spray serves as a carrier medium for the applied ultrasound energy which enhances the features and performance of the scalpel. Ultrasonic energy transmitted from the transducer tip assists the transport of the liquid to a liquid blade formed outside of the cavity. The ultrasound energy focuses and activates the liquid to allow cutting of tissue with the liquid blade.

Thus, according to a particular aspect of the present invention there is provided a method of producing a polymeric implantable medical device, said method comprising:
positioning a polymeric tube on a mandrel such that the mandrel is within the polymeric tube, e.g. is in contact with the inner surface of the polymeric tube;
rotating the polymeric tube about the mandrel;
simultaneously cutting and annealing the polymeric tube whilst the mandrel is positioned within the polymeric tube to form a polymeric implantable medical device with an opening; and
removing the polymeric implantable medical device from the mandrel.

The polymeric implantable medical device of the present invention may comprise a range of polymer materials suitable for use in the construction of implantable medical devices. Suitable polymer materials include both biodegradable and non-biodegradable plastic materials. In some applications, a polymeric biodegradable material may be preferred. Suitable polymer materials include, but shall not be limited to, silicon rubber, nitinol, nylon, polyurethane, polyethylene terephthalate (PETE), latex and thermoplastic elastomers. Silicone is one of the more common choices because it is inert and unreactive to body fluids and a range of medical fluids with which it might come into contact.

In one aspect of the invention the polymeric implantable medical device is not a urinary catheter.

Implantable medical devices include percutaneous implants in which a portion or the entire device is introduced or inserted into the body of a patient but that are not necessarily required to reside at the target location in the body for an extended period of time. Insertable medical devices can include those that are moved in the body, such as to deliver a fluid, drug, or an implantable medical device to a target location in the body.

Examples of implantable medical devices include, but shall not be limited to vascular implants and grafts, grafts, surgical devices; polymeric prostheses; vascular prosthesis including endoprosthesis, stent-graft, and endo-vascular-stent combinations; small diameter grafts, abdominal aortic aneurysm grafts; wound management device; haemostatic barriers; mesh and hernia plugs; ASD (atrial septal defect), PFO (patent foramen ovale) and VSD (ventricular septal defect) closures; percutaneous closure devices, valve repair devices; valve annuloplasty devices, catheters (other than urinary catheters); central venous access catheters, vascular access catheters, abscess drainage catheters, drug infusion catheters, parenteral feeding catheters, intravenous catheters (e.g. treated with an antithrombotic agent), stroke therapy catheters, blood pressure and stent graft catheters; anastomosis devices and anastomotic closures; aneurysm exclusion devices; biosensors including glucose sensors; cardiac sensors; birth control devices; breast implants; infection control devices; membranes; tissue scaffolds; tissue- related materials; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; orthopaedic devices such as orthopaedic joint implants, bone repair/augmentation devices, cartilage repair devices; urethral devices such as urological implants, bladder devices (other than urinary catheters), renal devices and hemodialysis devices, colostomy bag attachment devices; biliary drainage products.

In another aspect of the invention the polymeric implantable medical device is not a catheter.

In another aspect of the invention the polymeric implantable medical device is subcutaneous implant. Such subcutaneous implants are useful, inter alia, for sustained release of medicaments. For example, U.S. Pat. No. 5,004,602 discloses a process for the preparation of a drug delivery system comprising a solid implant using melt extrusion in which freeze dried drug, e.g. a peptide, such as goserelin acetate (Zoladex®) and polymer has been extruded together under pressure at 70° C. to form a rod, from which implants of the required weight can be cut.

For drug delivery systems a suitable biodegradable polymer may be a polylactide, e.g. polymers of lactic acid alone, copolymers of lactic acid and glycolic acid, mixtures of such polymers, mixtures of such copolymers, and mixtures of such polymers and copolymers, the lactic acid being either in racemic or in optically active form.

Thus, according to this aspect of the invention there is provided a method of manufacturing an implantable drug delivery device comprising simultaneously cutting and annealing an extruded slab of a drug/polymer material.

According to this aspect of the invention there is provided a polymeric implantable drug delivery device comprising one or more cut surfaces of low rugosity. Such drug delivery devices may be prepared by the method as hereinbefore described. The invention further provides the use of a polymeric implantable drug delivery device as hereinbefore described in the delivery of a medicament wherein the drug delivery device is manufactured by the simultaneous cutting and annealing of one or more cut surfaces.

A preferred polymeric implantable medical device is a stent.

The method of the invention is advantageous because, inter alia, it provides a method of manufacturing an implantable medical device provided with an opening wherein the surface adjacent the opening is of low rugosity.

Thus, according to a further aspect of the invention there is provided a polymeric implantable medical device comprising an opening wherein the surface adjacent the opening is of low rugosity.

The polymeric implantable medical device may comprise any of those implantable medical devices hereinbefore described.

By the term “low rugosity” is meant a rugosity lower than that of conventional polymeric implantable medical devices. Rugosity is generally a measure of surface roughness, therefore the lower the rugosity value, the smoother the surface. As hereinbefore described, the problem with prior art polymeric implantable medical devices is that, inter alia, the surface roughness at the edges of the opening is too high. Therefore, the present invention particularly provides a polymeric implantable medical device where the edges or rim of the opening are substantially smooth, i.e. of low rugosity.

The low rugosity opening of the polymeric implantable medical device of the invention is advantageous in that it is smooth and substantially non-adherent, e.g. non-adherent to bacterial and/or yeast cells.

There are a variety of methods available for the measurement of the rugosity of a surface, including contact and non-contact methods. An example of a contact method comprises the use of a profilometer. Non-contact methods include, but shall not be limited to, interferometry, confocal microscopy, focus variation, structured light, electrical capacitance, electron microscopy and photogrammetry. Measurement by electron microscopy, e.g. SEM, is especially preferred.

The rugosity of the opening is Ra, where Ra is the arithmetic average of the rugosity which may optionally be made from a variety of methods. However, the rugosity will generally be about 1 μm or less, e.g. Ra may be from about 0.0001 μm to about 1 μm.

According to a further aspect of the invention there is provided the use of a polymeric implantable medical device comprising a tube with an opening wherein the polymeric implantable medical device is manufactured by the simultaneous cutting and annealing of the opening in the tube.

According to a yet further aspect of the invention there is provided the use of a polymeric implantable medical device comprising an opening wherein the surface adjacent the opening is of low rugosity for inserting or implanting into a patient.

The invention will now be illustrated by way of example only and with reference to the accompanying figures. The figures illustrate that ultrasonically openings, e.g. cut eye holes compared with standard punched eye holes are much smoother with less tooling imperfections when viewed under the electron microscope. This smoother surface results in less E. coli cells adhering to the drainage opening “eye hole” surface over 72 hrs.

With reference to the figures:

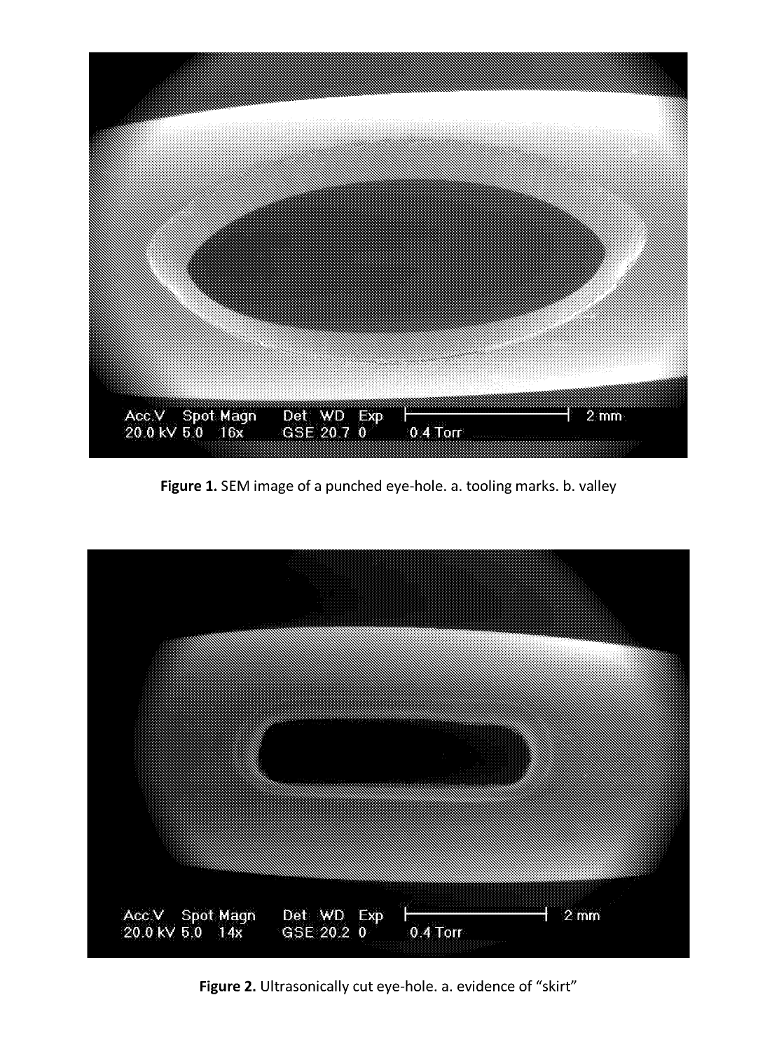

FIG. 1 is a Scanning Electron Microscope (SEM) image of a punched eye-hole illustrating a valley and tooling marks;

FIG. 2 is an SEM of an ultrasonically cut eye-hole illustrating evidence of “skirt”;

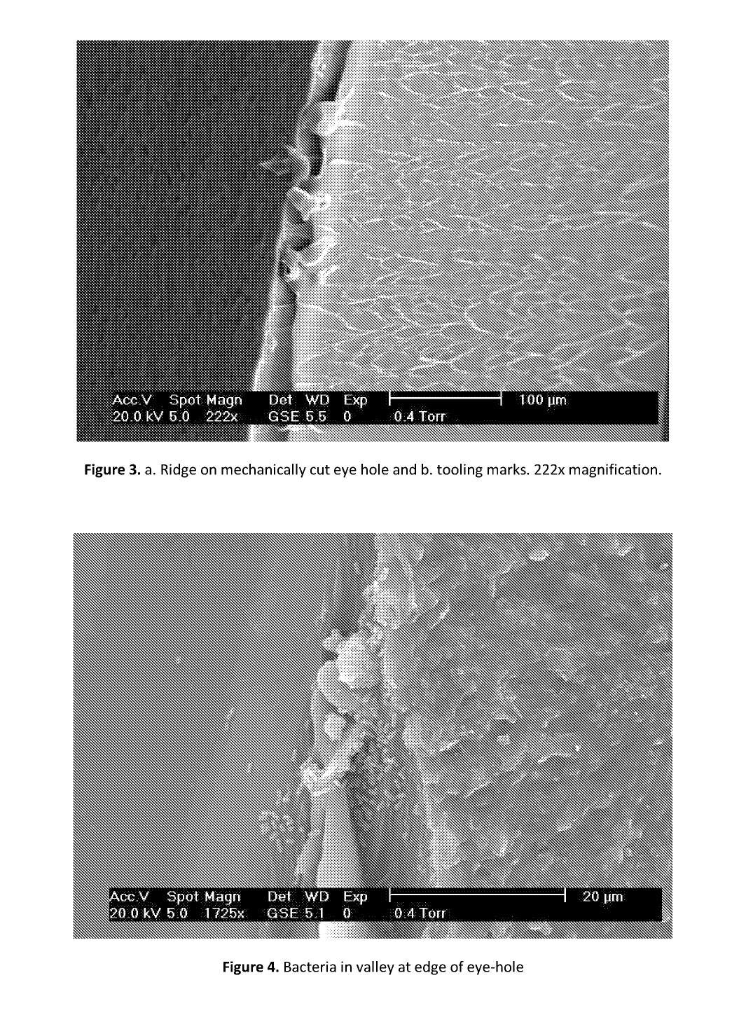

FIG. 3 is an SEM of a mechanically cut eye hole illustrating a ridge and tooling marks;

FIG. 4 is an SEM illustrating bacteria growth in a valley at the edge of mechanically cut eye-hole;

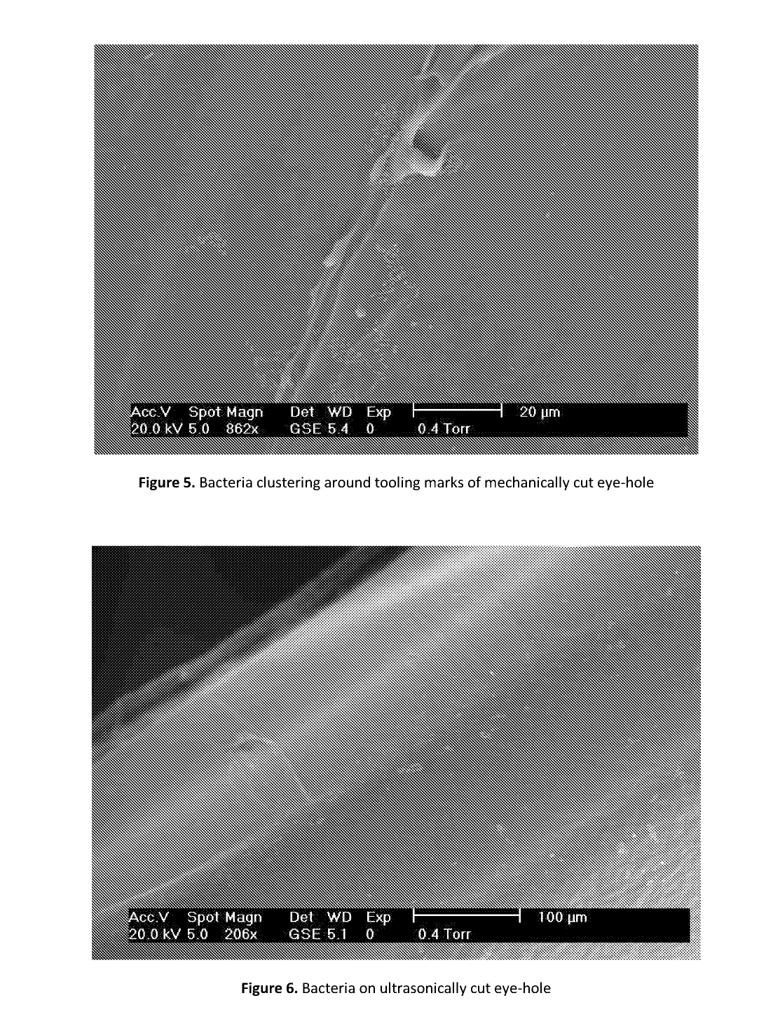

FIG. 5 is an SEM illustrating bacteria clustering around tooling marks of mechanically cut eye-hole;

FIG. 6 is an SEM illustrating bacteria on an ultrasonically cut eye-hole;

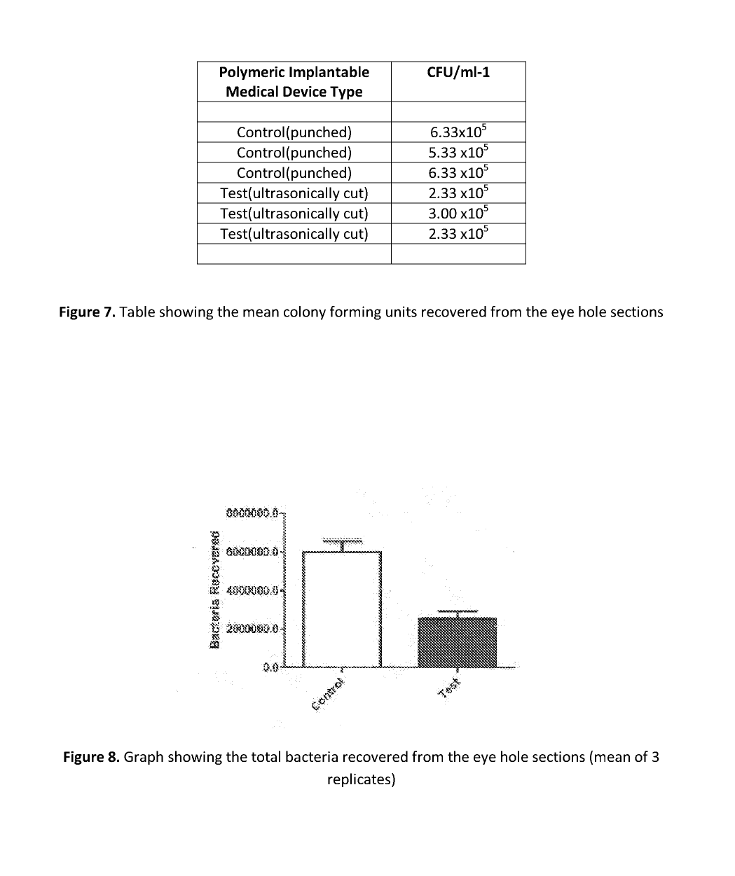

FIG. 7 is a table showing the mean colony forming units recovered from the eye hole sections;

FIG. 8 is a graph showing the total bacteria recovered from the eye hole sections (mean of 3 replicates);

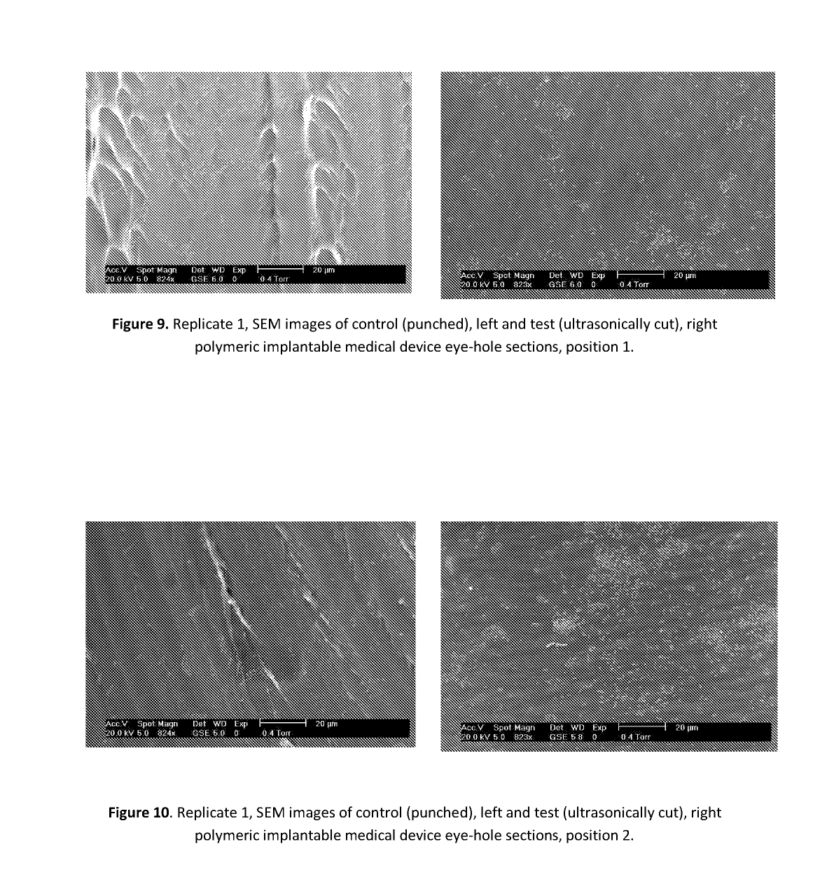

FIG. 9 is a pair SEM images for “Replicate 1” illustrating (left) a control (punched) polymeric implantable medical device eye-hole sections; and (right) a test (ultrasonically cut) polymeric implantable medical device eye-hole sections; taken from position 1;

FIG. 10 is a pair SEM images for “Replicate 1” illustrating (left) a control (punched) polymeric implantable medical device eye-hole sections; and (right) a test (ultrasonically cut) polymeric implantable medical device eye-hole sections; taken from position 2;

FIG. 11 is a pair SEM images for “Replicate 1” illustrating (left) a control (punched) polymeric implantable medical device eye-hole sections; and (right) a test (ultrasonically cut) polymeric implantable medical device eye-hole sections; taken from position 3;

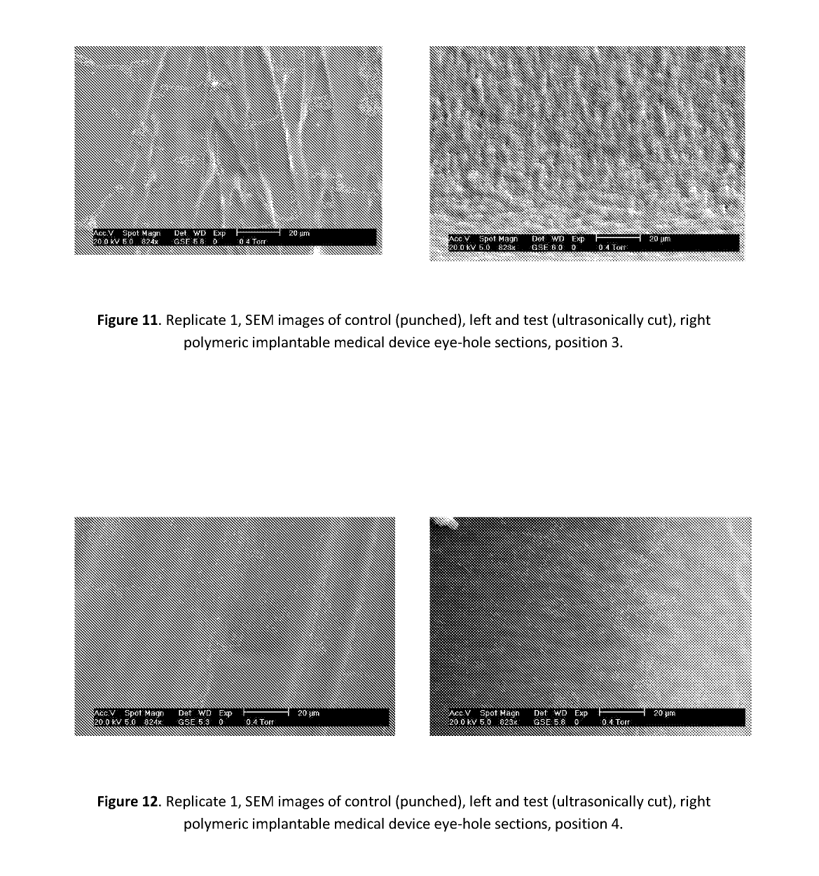

FIG. 12 is a pair SEM images for “Replicate 1” illustrating (left) a control (punched) polymeric implantable medical device eye-hole sections; and (right) a test (ultrasonically cut) polymeric implantable medical device eye-hole sections; taken from position 4;

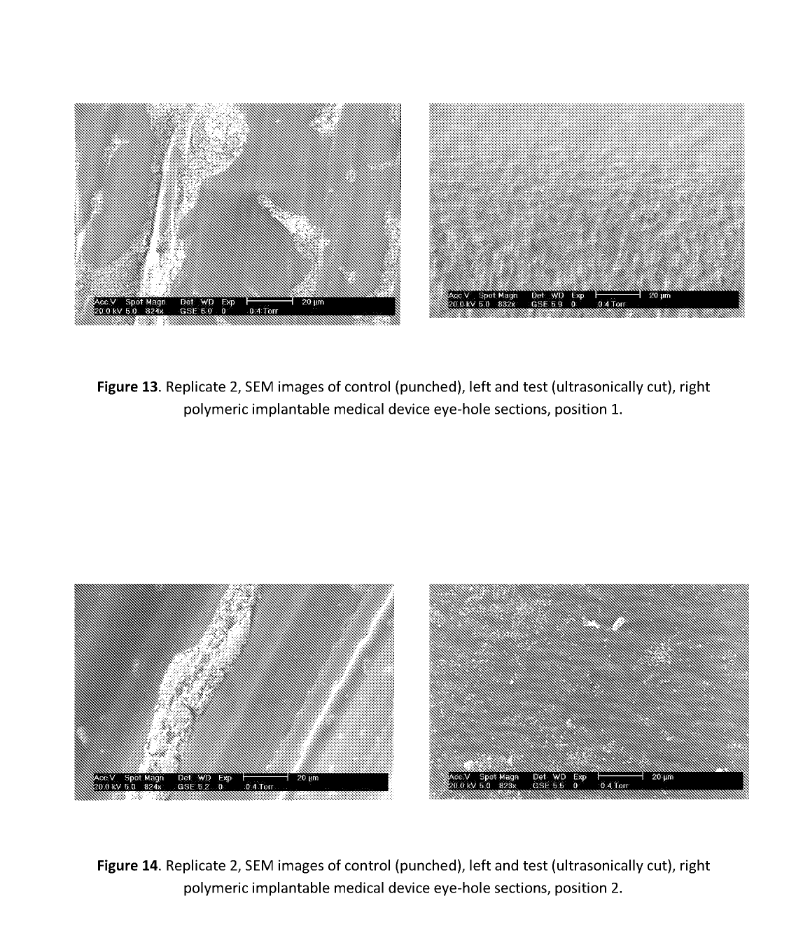

FIG. 13 is a pair SEM images for “Replicate 2” illustrating (left) a control (punched) polymeric implantable medical device eye-hole sections; and (right) a test (ultrasonically cut) polymeric implantable medical device eye-hole sections; taken from position 1;

FIG. 14 is a pair SEM images for “Replicate 2” illustrating (left) a control (punched) polymeric implantable medical device eye-hole sections; and (right) a test (ultrasonically cut) polymeric implantable medical device eye-hole sections; taken from position 2;

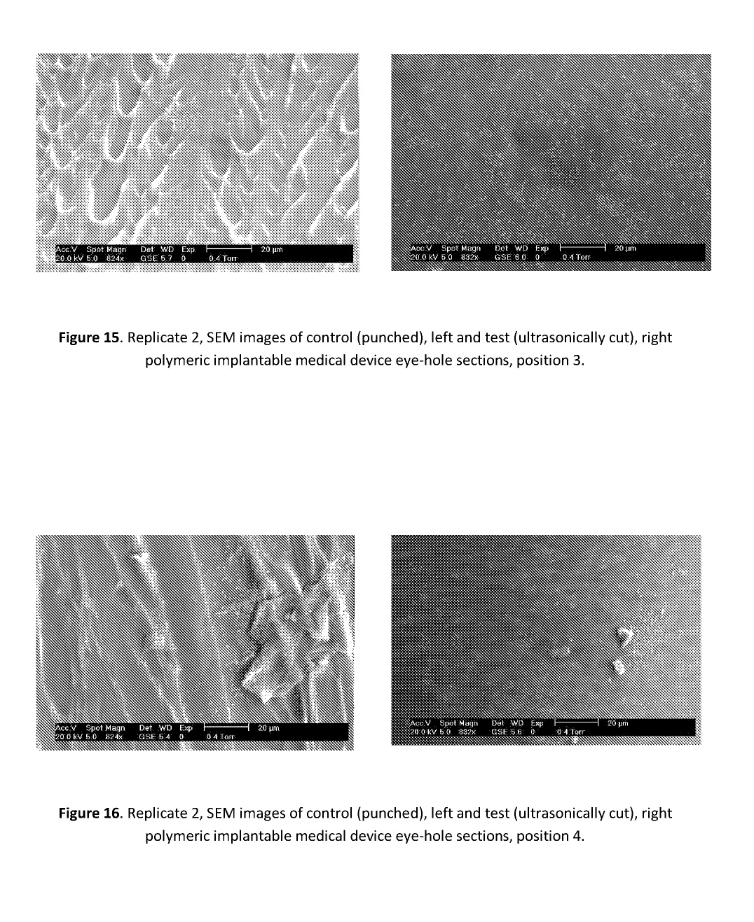

FIG. 15 is a pair SEM images for “Replicate 2” illustrating (left) a control (punched) polymeric implantable medical device eye-hole sections; and (right) a test (ultrasonically cut) polymeric implantable medical device eye-hole sections; taken from position 3;

FIG. 16 is a pair SEM images for “Replicate 2” illustrating (left) a control (punched) polymeric implantable medical device eye-hole sections; and (right) a test (ultrasonically cut) polymeric implantable medical device eye-hole sections; taken from position 4;

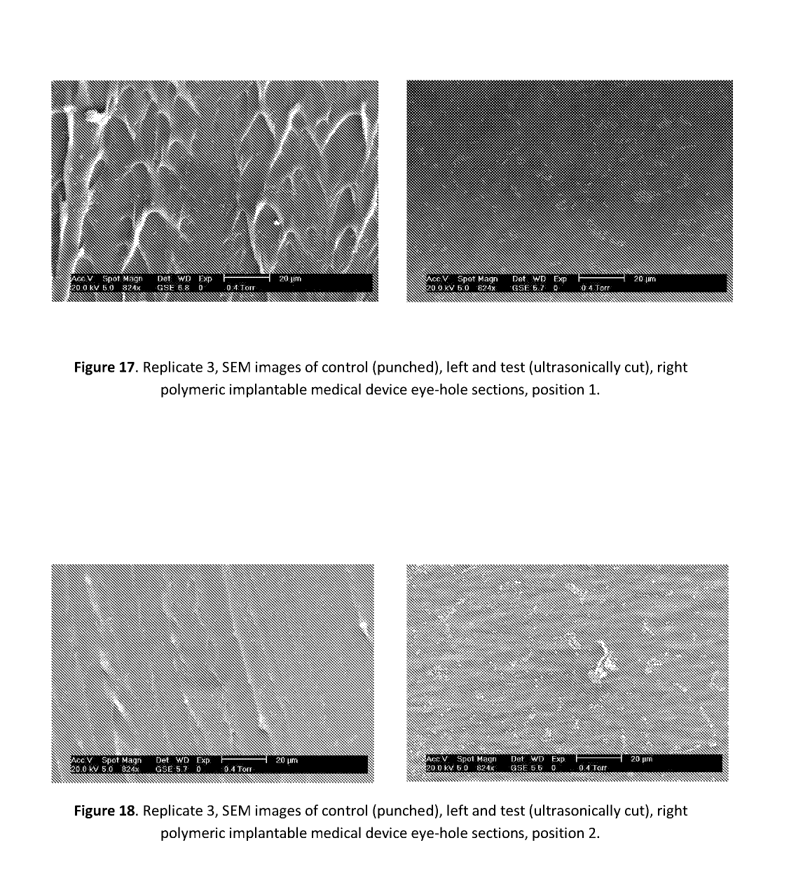

FIG. 17 is a pair SEM images for “Replicate 3” illustrating (left) a control (punched) polymeric implantable medical device eye-hole sections; and (right) a test (ultrasonically cut) polymeric implantable medical device eye-hole sections; taken from position 1;

FIG. 18 is a pair SEM images for “Replicate 3” illustrating (left) a control (punched) polymeric implantable medical device eye-hole sections; and (right) a test (ultrasonically cut) polymeric implantable medical device eye-hole sections; taken from position 2;

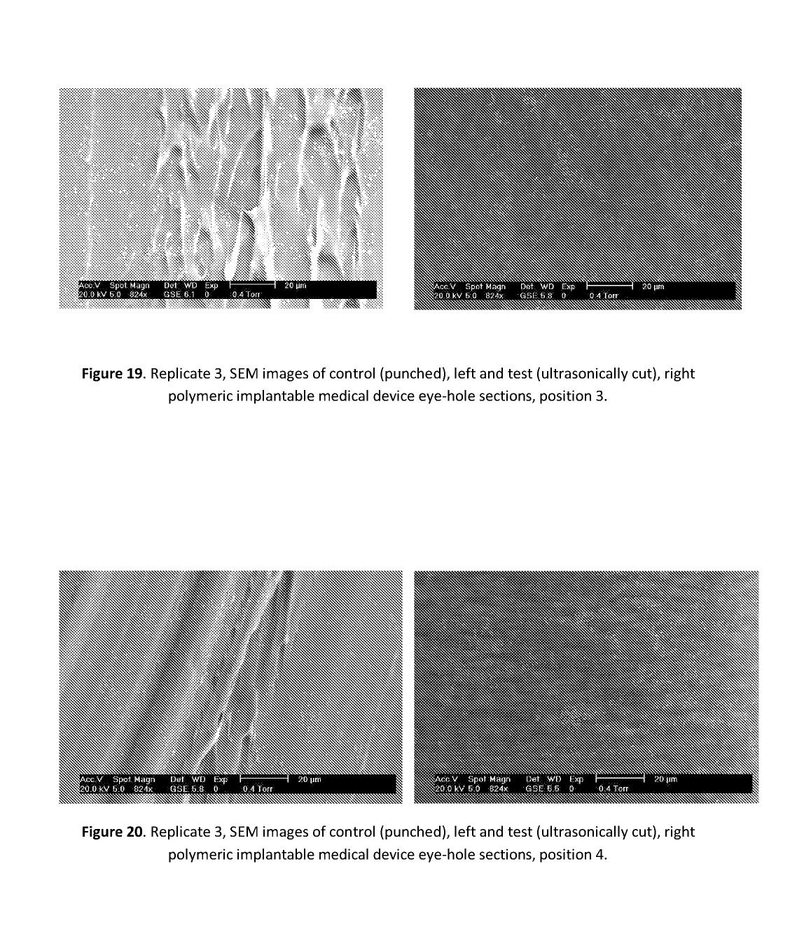

FIG. 19 is a pair SEM images for “Replicate 3” illustrating (left) a control (punched) polymeric implantable medical device eye-hole sections; and (right) a test (ultrasonically cut) polymeric implantable medical device eye-hole sections; taken from position 3; and

FIG. 20 is a pair SEM images for “Replicate 3” illustrating (left) a control (punched) polymeric implantable medical device eye-hole sections; and (right) a test (ultrasonically cut) polymeric implantable medical device eye-hole sections; taken from position 4.

1. A method of manufacturing a polymeric implantable medical device wherein the medical device comprises a tube with an opening said method comprising the simultaneous cutting and annealing of the opening.

2. A method of manufacturing a polymeric implantable medical device according to claim 1 wherein said method comprises ultrasonically cutting an opening in the polymeric implantable medical device.

3. A method according to claim 1 which comprises the use of an ultrasonic drill or scalpel.

4. A method according to any one of the preceding claim 1 which comprises positioning a polymeric tube on a mandrel such that the mandrel is within the polymeric tube, e.g. is in contact with the inner surface of the polymeric tube;
rotating the polymeric tube about the mandrel;
simultaneously cutting and annealing the polymeric tube whilst the mandrel is positioned within the polymeric tube to form a polymeric implantable medical device with an opening; and
removing the polymeric implantable medical device from the mandrel.

5. A method according to claim 1 wherein the polymeric implantable medical device is not a urinary catheter.

6. A method according to claim 1 wherein the polymeric implantable medical device is a stent.

7. A method according to claim 1 wherein the method produces an implantable medical device provided with an opening wherein the surface adjacent the opening is of low rugosity.

8. A polymeric implantable medical device comprising an opening wherein the surface adjacent the opening is of low rugosity.

9. A polymeric implantable medical device according to claim 8 wherein the edges or rim of the opening are substantially smooth.

10. A polymeric implantable medical device according to claim 8 wherein the edges or rim of the opening are substantially non-adherent to bacterial and/or yeast cells.

11. A polymeric implantable medical device according to claim 8 wherein the Ra is about 1 μm or less.

12. A polymeric implantable medical device according to claim 8 wherein the Ra is from about 0.0001 μm to about 1 μm.

13. A polymeric implantable medical device according to claim 8 wherein the polymeric implantable medical device comprises a range of polymer materials selected from silicon rubber, nitinol, nylon, polyurethane, polyethylene terephthalate (PETE), latex and thermoplastic elastomers.

14. A polymeric implantable medical device according to claim 8 wherein the polymeric implantable medical device is not a urinary catheter.

15. A polymeric implantable medical device according to claim 8 wherein the polymeric implantable medical device is a stent.

16. (canceled)

17. (canceled)

18. A method of manufacturing an implantable drug delivery device comprising simultaneously cutting and annealing an extruded slab of a drug/polymer material.

19. A polymeric implantable drug delivery device according to claim 8 wherein the polymeric implantable drug delivery device comprises one or more cut surfaces of low rugosity.

20. (canceled)

21. (canceled)
